(19) United States
(12) Patent Application Publication
(10) Pub. No.: US 2008/0311587 A1
(43) Pub. Date: Dec. 18, 2008

(54) Use of Genetic Information to Detect a Predisposition for Bone Density Conditions

(75) Inventor: Robert P. Ricciardi, Kennet Square, PA (US)

Correspondence Address: BRANDY C HILL, P.O. BOX 951121, LAKE MARY, FL, 32795, US

(73) Assignee: GENELINK, INC., Longwood, FL (US)

(21) Appl. No.: 12/198,424

(22) Filed: Aug. 26, 2008

Related U.S. Application Data
(63) Continuation-in-part of application No. 09/924,011, filed on Aug. 7, 2001, now abandoned.

(51) Int. Cl. C12Q 1/68

(52) U.S. Cl. 435/6

(57) ABSTRACT

The invention relates to kits and methods for assessing susceptibility of a human to an undesirable bone density condition, such as osteopenia and osteoporosis, and advising appropriate interventions. The methods involve contemporaneously assessing occurrence in the human's genome of a plurality of polymorphisms (e.g., single nucleotide polymorphisms) that occur in one or more genes associated with bone density regulation and that are associated with a disorder in humans. Preferred assessment and scoring methods are disclosed, as are kits for performing the methods.

### CROSS-REFERENCE TO RELATED APPLICATIONS

This application is a Continuation-in-Part of U.S. patent application Ser. No. 09/924,011 filed Aug. 7, 2001, now pending and expressly incorporated herein in its entirety, and is entitled to priority pursuant to 35 USC § 120 and 37 CFR § 1.78.

### STATEMENT REGARDING FEDERALLY SPONSORED RESEARCH

Not applicable.

### REFERENCE TO A MICROFICHE APPENDIX

Not applicable.

### BACKGROUND OF THE INVENTION

Many undesirable bone density conditions can be alleviated, inhibited, or even prevented by maintaining a high degree of health or by timely intervention, including administration of appropriate compositions which modulate bone density (e.g., a composition comprising a compound that modulates parathyroid hormone release, modulates binding of parathyroid hormone to a parathyroid hormone receptor, or modulates the response induced upon binding of parathyroid hormone receptor with parathyroid hormone). Such intervention can further include advising consumption or consuming a customized (i.e., genetically-guided nutrigenomic) or generic nutritional or pharmaceutical composition known to be effective against undesirable bone density conditions or advising or undergoing heightened medical monitoring. These changes are often not made, owing to the expense or inconvenience of the changes and the individual's subjective belief that he or she is not at high risk for developing an undesirable bone density condition. Improved assessment of undesirable bone density conditions can help identify individuals at risk for developing an undesirable bone density condition and permit more informed decisions to be made regarding whether lifestyle changes or other interventions are justified.

Osteoporosis, a disease characterized by porous bones, is a serious public health concern. Patients afflicted with osteoporosis exhibit low bone density, structural deterioration of bone tissue, and a high susceptibility to bone fracture. Osteoporosis patients also exhibit complications such as disability, decreased quality of life, and increased likelihood of mortality.

Four diagnostic categories regarding bone density have been identified, i) normal, ii) osteopenia, iii) osteoporosis, and iv) established osteoporosis. These four categories can be differentiated based on bone density and the presence of fractures. Osteopenia is defined as bone density that is somewhat low, i.e., between one and 2.5 standard deviations below average bone density for young, healthy individuals. Osteopenia is often not characterized by symptoms discemable to the patient, but is clinically detectable as a precursor to osteoporosis. Osteoporosis is defined as bone density that is at least 2.5 standard deviations below average density for young, healthy individuals. Low bone density can result from decreased bone formation, increased bone erosion and resorption, or a combination of these factors.

Bone tissue is primarily composed of three cell types (osteoblasts, osteocytes, and osteoclasts) and a mineralized intercellular bone matrix comprising polymers (primarily collagen fibers) and other organic substances (ground substance, composed primarily of proteoglycans such as chondroitin sulfate and hyaluronic acid) synthesized by bone cells (primarily osteoblasts). Bone cells produce the organic molecules of bone matrix and also modulate its mineralization. Osteoblasts are located at bone tissue surfaces and synthesize the organic components of the bone matrix. Osteocytes are mature osteoblasts and are involved in maintaining the bone matrix. Osteoclasts are involved in bone erosion and resorption. Bone erosion and resorption are normal processes that the body uses to maintain constant levels of ions such as calcium and phosphate in bodily fluids. In a normal individual, the process of bone formation and the process of bone erosion and resorption are in a state of on-going balance, both processes occurring constantly, whereby normal bone density is maintained.

Maintaining normal bone density can be difficult for those susceptible to an undesirable bone density condition such as osteopenia or osteoporosis. The biochemical elements of an individual's bone formation, erosion, and resorption processes affect bone density regulation in the individual, and the relative degrees of expression and activity of those elements among individuals can account for differences in bone density that are not clearly attributable to any particular disease or disorder. Thus, the ability to characterize differences in the biochemical elements involved in bone density regulation among individuals would permit individualization of treatment, behavioral modifications, nutritional supplementation, and other processes that can limit, inhibit, prevent, or reverse morbidity and mortality associated with undesirable bone density conditions.

Maintenance of normal bone density is known to be influenced by the products of several genes. For example, parathyroid hormone (PtH) and calcitonin are proteins known to be involved in regulation of bone density. Differences in activity or expression of genes involved in bone density regulation can influence a person's bone mass and density.

PtH, a polypeptide hormone that exists in a variety of forms having about 34 to 84 amino acid residues, binds with one or more PtH receptors on the surface of osteoblasts and osteocytes. Binding of PtH and its receptor enhances bone resorption in at least two ways. PtH rapidly enhances release of mineralized calcium and phosphate from bone into extracellular fluid. Over a longer span of time, PtH enhances proliferation of osteoclasts. Because PtH receptors are apparently not expressed on osteoclasts, the rapid effect of PtH on bone demineralization appears to be mediated by an activating effect of a second messenger on osteoclasts, by enhancing passage of calcium and phosphate through the osteocytic membrane system (which surrounds bone and interconnects osteoblasts and osteocytes), or by some combination of these two mechanisms. PtH also decreases calcium excretion in the urine, and enhances phosphate excretion in the urine. Thus, PtH promotes erosion and resorption of bone matrix and decreases bone density.

Calcitonin, a 32-residue polypeptide hormone, binds with a calcitonin receptor and enhances bone formation. The effects of calcitonin on bone formation appear to have at least two components. Calcitonin enhances calcium uptake by decreasing bone absorption by osteoclasts, by enhancing calcium uptake and/or retention by the osteocytic membrane system, or both. Calcitonin also decreases the rate at which new osteoclasts are formed. Thus, calcitonin inhibits erosion and resorption of bone matrix and increases bone density.

Most, if not all, human genes occur in a variety of forms which differ in at least minor ways. Heterogeneity in human genes is believed to have arisen, in part, from minor, non-fatal mutations that have occurred in the genome over time. In some instances, differences between alternative forms of a gene are manifested as differences in the amino acid sequence of a protein encoded by the gene. Some amino acid sequence differences can alter the reactivity, substrate specificity, or inter-protein binding specificity of the protein. Differences between alternative forms of a gene can also affect the degree to which (if at all) the gene is expressed. However, many heterogeneities that occur in human genes appear not to be correlated with any particular phenotype. Known heterogeneities include, for example, single nucleotide polymorphisms (i.e., alternative forms of a gene having a difference at a single nucleotide residue). Other known polymorphic forms include those in which the sequence of larger (e.g., 2-1000 residues) portions of a gene exhibits multiple sequence differences and those which differ by the presence or absence of portion of a gene.

Numerous disorders and physiological states have been correlated with occurrence of one or more alternative forms of a gene in the genome of a human who exhibits the disorder or physiological state. For example, Kimura, et al. (2000, Am. J. Ophthalmol. 130:769-773) discloses an association between occurrence of a SNP of the manganese superoxide dismutase gene and a form of macular degeneration. As another example, Mammes et al. (2001, Eur. J. Clin. Invest. 31(5):398-404) reports a relationship between LEPR gene polymorphisms and common obesity phenotypes. Although associations between individual disorders and individual genetic polymorphisms are known, a need remains for a method of assessing the overall state of bone density regulation in a human and for a method of assessing a person's predisposition to develop an undesirable bone density condition.

### BRIEF SUMMARY OF THE INVENTION

The invention relates to a method of assessing relative susceptibility of a human to an undesirable bone density condition such as osteoporosis or osteopenia. This method comprises assessing occurrence in the human's genome of two or more disorder-associated polymorphisms (e.g., single nucleotide polymorphisms [SNPs] or di-, tri-, or tetra-nucleotide repeats) selected from the group consisting of

a) a polymorphism manifested as occurrence of a cytosine residue in the codon of the gene encoding transforming growth factor beta 1 protein corresponding to amino acid residue 10 of the protein, whereby the codon encodes proline;

b) a polymorphism manifested as occurrence of a thymine residue 8 residues upstream of the normal start codon of the gene encoding vitamin D receptor, whereby the residue is part of an initiation codon and the gene encodes a variant vitamin D receptor comprising three additional amino acids at its amino terminus;

c) a polymorphism manifested as occurrence of a nucleotide residue that is characteristic of apolipoprotein E polymorphic variant 4;

d) a polymorphism manifested as occurrence of a thymine residue in the gene encoding the alpha 1 subunit of type 1 collagen at a site which a guanine usually occurs, whereby a recognition site for the transcription of factor Sp1 is altered;

e) a polymorphism manifested as occurrence of a cytosine residue at position −174 of the interleukin 6 gene promoter;

f) a polymorphism manifested as occurrence of a guanine residue at the position which a cytosine residue normally occurs in the codon corresponding to amino acid residue 986 of the calcium sensing receptor gene, whereby the codon encodes a serine residue;

g) a polymorphism manifested as occurrence of a thymine residue at the position corresponding to position +1417 of the cDNA encoding PtH receptor;

h) a polymorphism manifested as occurrence of a thymine residue at the position at which a cytosine residue normally occurs in the codon corresponding to amino acid residue 447 of the calcitonin receptor gene, whereby the codon encodes a leucine residue;

i) a polymorphism manifested as occurrence of a thymine residue at amino acid position +1377 of the calcitonin receptor gene; and,

j) a polymorphism manifested as occurrence of a cytosine residue where a guanine residue normally occurs at the first nucleotide position of intron 2 of the PtH gene.

Occurrence of any of the aforementioned disorder-associated polymorphisms is an indication that the human is more susceptible to an undesirable bone density condition than a human whose genome does not comprise the polymorphism. An undesirable bone density condition can be manifested, for example, as occurrence in the human of a readily-detectable condition at an early stage (e.g., osteopenia) or at a late stage (e.g., osteoporosis) or as occurrence in the human of a propensity to lose bone density.

The method by which occurrence of an individual disorder-associated polymorphism is assessed is not critical. For example, occurrence of the polymorphisms can be assessed using a method that includes contacting a nucleic acid derived from the human's genome with a first oligonucleotide. The first oligonucleotide can be one that anneals with higher stringency with the disorder-associated polymorphism than with a corresponding non-disorder-associated polymorphism. Annealing of the first oligonucleotide and the nucleic acid can be assessed, and such annealing is an indication that the human's genome comprises the disorder-associated polymorphism. Use of an oligonucleotide has the advantage that the oligonucleotide can be attached to a support using routine methods, and that a plurality of oligonucleotides can be attached to the same support, to allow simultaneous detection of multiple polymorphisms. If a second oligonucleotide which anneals with higher stringency with a non-disorder-associated polymorphism than with a corresponding disorder-associated polymorphism is used, then the allelic content (i.e., heterozygous or homozygous for one or the other polymorphic form) of the human's genome can be determined. Detection of polymorphic sequences can be simplified by using labeled oligonucleotides, such as molecular beacon oligonucleotides.

Once the content of the human's genome for disorder-associated polymorphisms has been assessed, assessment of susceptibility to an undesirable bone density condition can further comprise calculating a susceptibility score for the human. A susceptibility score can be calculated by summing, for each of the disorder-associated polymorphisms that occurs in the human's genome, the product of a constant and a correlation factor. The correlation factor can, for example, be a factor that represents the fraction of humans heterozygous for the disorder-associated polymorphism who exhibit the corresponding disorder or a factor that represents the fraction of humans homozygous for the disorder-associated polymorphism who exhibit the corresponding disorder. The constant can be the same for each polymorphism, or it can be selected based on the known or surmised relevance of the corresponding gene with respect to bone formation, bone erosion, and bone resorption. The susceptibility score represents the relative susceptibility of the human to an undesirable bone density condition.

In another aspect, the invention relates to a method of selecting or advising a dose of a composition (whether customized, i.e., a genetically-guided nurtigenomic product, or generic) or composition which modulates bone density or affects the body's regulation of bone density (e.g., a composition comprising a compound that modulates parathyroid hormone release, modulates binding of parathyroid hormone to a parathyroid hormone receptor, or modulates the response induced upon binding of parathyroid hormone receptor with parathyroid hormone). Thus, this method can be used to identify compositions for administration to a human who exhibits, or is at risk for developing, an undesirable bone density condition. This method comprises assessing occurrence in the human's genome of disorder-associated polymorphisms as indicated above. After assessing occurrence of the polymorphisms, a dose of the composition is selected. Occurrence of any of the polymorphisms is generally an indication that a greater dose of the composition should be administered to the human in whom the disorder-associated polymorphism occurs than to a human in whom the disorder-associated polymorphism does not occur.

The invention also relates to a kit for assessing relative susceptibility of a human to an undesirable bone density condition. The kit comprises reagents for assessing occurrence in the human's genome of disorder-associated polymorphisms in at least one gene selected from the groups indicated above. Examples of suitable reagents include oligonucleotides (e.g., molecular beacon oligonucleotides) that anneal with higher stringency with the disorder-associated polymorphisms than with corresponding non-disorder-associated polymorphisms and oligonucleotide primers that are complementary to the region adjacent a characteristic residue of the disorder-associated polymorphism. These primers are useful for amplifying at least the characteristic residue, thereby facilitating its detection. The kit can further comprise an instructional material which includes a numerical value representing the product of a constant and a correlation factor for some or all of the disorder-associated polymorphisms.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. The invention is not limited to the precise arrangements and instrumentalities shown.

FIGS. 1A and 1B are images which depict examples of results that can be obtained by analyzing occurrence of polymorphisms in several genes. The results shown in FIG. 1A are derived from a hypothetical first human, and those shown in FIG. 1B are derived from a hypothetical second human. Circles represent different polymorphisms of the gene indicated to the left of the row of circles. Filled circles indicate the presence of the polymorphism. Non-filled circles indicate the absence of the polymorphism. Numbers below each circle represent a correlation factor for the polymorphism and a disease or disorder. The calculations to the right of the drawings represent 1) the sum of the number of polymorphisms present in the human's genome and 2) the total correlation factor.

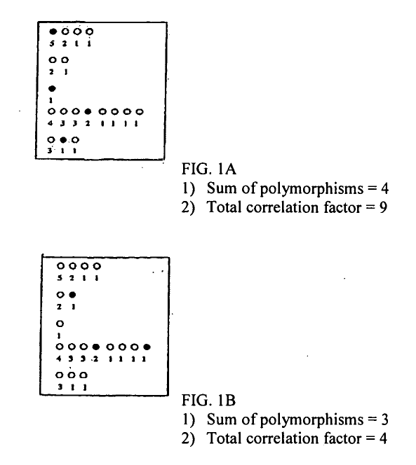

FIG. 2 is an image which depicts an example of an assessment of a hypothetical human's genome for the polymorphisms assessed by the inventor's methods. The column to the left identifies the polymorphism assessed and the column to the right indicates the presence of an advantaged allele with a (−) sign, i.e., absence of the polymorphic allele, and the presence of a disadvantaged allele with a (+) sign, i.e., occurrence of a polymorphic allele. An individual who is homozygous negative for the polymorphic form of the gene is denoted (−/−) and exhibits optimum gene function with respect to the assessed polymorphism and requires basic support from a composition, nutritional supplement, composition which modulates bone density, or other intervention. An individual who is heterozygous for the polymorphic form of the gene is denoted (+/−) and exhibits less than optimal gene function with respect to the assessed polymorphism and requires added support from a composition, nutritional supplement, composition which modulates bone density, or other intervention. An individual who is homozygous positive for the polymorphic form of the gene is denoted (+/+) and exhibits minimal gene function with respect to the polymorphism assessed and requires maximum support from a composition, nutritional supplement, composition which modulates bone density, or other intervention.

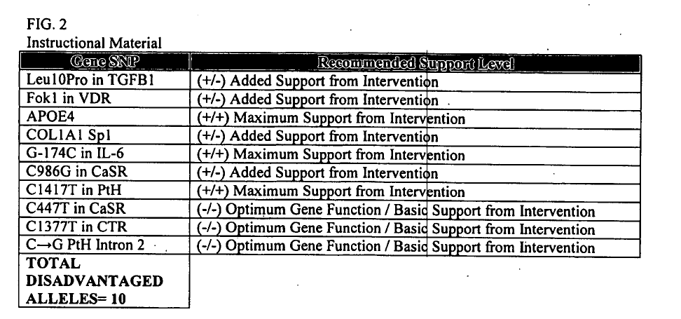

TABLE 1 lists each of the polymorphisms assessed in the left-hand column, denotes the advantaged allele in the center column, and indicates the disadvantaged allele in the right-hand column.

### DETAILED DESCRIPTION OF THE INVENTION

The invention relates to kits and methods for assessing the relative susceptibility of a human to undesirable bone density conditions such as osteoporosis and osteopenia. Undesirable bone density conditions are physiological conditions characterized by occurrence of abnormal bone density (i.e., abnormally high or, more commonly, abnormally low bone density). The susceptibility or propensity of a human to develop an undesirable bone density condition can be determined by assessing occurrence in certain genes of genetic polymorphisms that are associated with disorders (not necessarily bone density disorders). Crudely simplified, the methods involve determining whether one or more polymorphisms that have been associated (by the inventor or by others) with a disorder (e.g., a disease or pathological state) in humans occur in a gene encoding a product associated with maintenance of bone density. Examples of such products include proteins that regulate or participate in bone formation, bone erosion, or bone resorption.

In some embodiments, the number of polymorphisms that occur in the human's genome are summed to yield a value; the higher the value is, the greater the susceptibility of the human to an undesirable bone density condition is assessed to be. A summed value greater than zero indicates that the human exhibits an increased susceptibility to development of an undesirable bone density condition, even in a human who is asymptomatic of any undesirable bone density condition. The increased susceptibility occurs whether the assessed polymorphisms are rare or common within certain populations (e.g., ethnic groups) because the methods assess relative susceptibility. In other embodiments, a weighting factor is assigned to each polymorphism tested, and the weighting factors of polymorphisms that occur in the human's genome are summed to yield a value that represents relative susceptibility to an undesirable bone density condition. The weighting factor can, for example, represent the product of a constant assigned to the gene in which the corresponding polymorphism occurs and a correlation factor that describes how informative an occurrence of the polymorphism is for occurrence of the disorder with which it is associated (again, this disorder need not be a bone density disorder). The invention includes a variety of alternative methods and kits for performing the methods, as described in greater detail herein.

### Definitions

As used in this disclosure, the following terms have the meanings associated with them in this section.

A “polymorphism” in a gene is one of the alternative forms of a portion of the gene that are known to occur in the human population. For example, many genes are known to exhibit single nucleotide polymorphic forms, whereby the identity of a single nucleotide residue of the gene differs among the forms. Each of the polymorphic forms represents a single polymorphism, as the term is used herein. Other known polymorphic forms include alternative forms in which multiple consecutive or closely-spaced, non-consecutive nucleotide residues vary in sequence, forms which differ by the presence or absence of a single nucleotide residue or a small number of nucleotide residues, and forms which exhibit different mRNA splicing patterns.

A “single nucleotide polymorphism” (“SNP”) is one of the alternative forms of a portion of a gene that vary only in the identity of a single nucleotide residue in that portion.

A “disorder-associated” polymorphism is an alternative form of a portion of a gene, wherein occurrence of the alternative form in the genome of a human has been correlated with exhibition by the human of a disease or a pathological state. Each of the assessed polymorphisms is a disorder-associated polymorphism.

A “non-disorder-associated” polymorphism is an alternative form of a portion of a gene for which no significant correlation has been made between occurrence of the alternative form in the genome and a disease or a pathological state. Non-disorder-associated polymorphisms are sometimes designated “neutral” polymorphisms in the art.

A disorder-associated polymorphism and a non-disease-associated polymorphism “correspond” with one another if the two polymorphisms are two alternative forms of the same portion of the gene. By way of example, if the identity of residue 100 of a gene is adenine in a disorder-associated polymorphism of the gene and cytosine in a non-disorder-associated polymorphism of the gene, then the two polymorphisms correspond with one another. It is understood that there may be three or more corresponding polymorphisms when there are more than two alternative forms of the same portion of the gene. When a disorder has multiple corresponding polymorphisms associated with it, the polymorphism having the lowest correlation with the disorder (i.e., the polymorphic form which occurs least frequently in humans afflicted with the disorder) is a non-disorder-associated polymorphism, and the other polymorphic forms are disorder-associated polymorphisms if they occur significantly more frequently among humans afflicted with the disorder.

A “characteristic residue” of a polymorphism is a nucleotide residue, the identity of which is known to vary among the alternative forms corresponding to the polymorphism.

A “undesirable bone density condition” is a physiological state associated with abnormal bone density or aberrant regulation of bone density. Abnormal bone density includes both abnormally low bone density (such as that associated with osteoporosis and osteopenia) and abnormally high bone density (such as that associated with osteopetrosis). Other undesirable bone density conditions which may be indicated by the methods described herein include decreased bone metabolism, osteomalacia and osteitis deformans. An individual exhibits abnormal bone density or aberrant regulation of bone density if the individual exhibits a physiological state wherein the degree or rapidity of at least one process selected from the group consisting of bone formation, bone erosion, and bone resorption signaling differs significantly (i.e., by at least 10%, 25%, 50%, 100%, 200%, or 500% or more) from the same process in a normal individual.

A “molecular beacon oligonucleotide” is a single-stranded oligonucleotide having a fluorescent label (e.g., rhodamine, FAM, TET, VIC, JOE, or HEX) attached to or near one end thereof and a fluorescence quencher (e.g., TAMRA or DABCYL) attached to or near the other end thereof, as described (Kostrikis et al., 1998, Science 279:1228-1229).

Two molecular beacon oligonucleotides are “spectrally distinct” if they can be differentially detected using spectrophotometric or spectrofluorimetric methods. Examples of characteristics that can be used to differentiate spectrally distinct oligonucleotides include absorption or excitation wavelength, emission wavelength, and fluorescent lifetime.

An “instructional material” is a publication, a recording, a diagram, or any other medium of expression which can be used to communicate how to use a kit described herein, numerical values for weighting the significance of various polymorphisms that are detectable using the kit, numerical values or symbolic representations for communicating the presence or absence of the polymorphisms assessed in the human's genome, or all of the aforementioned. The instructional material of the kit of the invention can, for example, be affixed to a container which contains a kit of the invention or be shipped together with a container which contains the kit. Alternatively, the instructional material can be shipped separately from the container with the intention that the instructional material and the kit be used cooperatively by the recipient.

The “stringency” with which two polynucleotides anneal means the relative likelihood that the polynucleotides will anneal in a solution as the conditions of the solution become less favorable for annealing. Examples of stringent conditions are known in the art and can be found in available references (e.g., Current Protocols in Molecular Biology, John Wiley & Sons, N.Y., 1989, 6.3.1-6.3.6). Aqueous and non-aqueous annealing methods are described in that reference and either can be used. In general, a first pair of polynucleotides anneal with higher stringency than a second pair if the first pair is more likely to anneal (or remain annealed) as one or more of the salt concentration, temperature, and detergent concentration are increased. With respect to a disorder, a “correlation factor” for a disorder-associated polymorphism is the fractions of humans who are heterozygous or homozygous for the polymorphism who exhibit the disorder. The correlation factor can, alternatively, be based solely on those who are heterozygous, solely on those who are homozygous, or on those who are either heterozygous or homozygous.

A “non-extendable” nucleotide residue is a nucleotide residue that is capable of being added to a polynucleotide by a polymerase (i.e., by extension of the polynucleotide in association with a complement thereof, catalyzed by the polymerase) and that, upon addition to the polynucleotide, renders the polynucleotide incapable of being further extended by the polymerase.

A “composition” is a compound comprising a nutritional (whether customized, i.e., a genetically-guided nutrigenomic product, or generic) or pharmaceutical composition known to be effective against undesirable bone density conditions

A “composition that [which] modulates bone density” (also a “composition”) comprises a compound that modulates parathyroid hormone release, modulates binding of parathyroid hormone to a parathyroid hormone receptor, or modulates the response induced upon binding of parathyroid hormone receptor with parathyroid hormone). Such composition that modulates bone density can include a nutritional or pharmaceutical composition known to be effective against undesirable bone density conditions

An “intervention” is advising or employing consumption of a nutritional or pharmaceutical composition known to be effective against undesirable bone density conditions (i.e., a composition), advising or employing a composition that modulates bone density, or advising or undergoing heightened medical monitoring.

A “nutrigenomic” composition is a customized nutritional supplement formulated to address an individual's supplementation needs based on the human's predisposition to certain conditions. Nutrigenomic compositions non-pharmaceutical; they are, however, compensatory and can be used to alleviate, inhibit, or prevent a disease state.

### Best Mode of Practicing the Invention

Bone density conditions such as osteoporosis are polygenic diseases whose determining loci have yet to be identified. Crudely simplified, the methods involve determining whether one or more polymorphisms that have been associated (by the inventor or by others) with a disorder (e.g., a disease or pathological state) in humans occur in a gene encoding a product associated with maintenance of bone density. Examples of such products include proteins that regulate or participate in bone formation, bone erosion, or bone resorption. Identification of the polymorphisms assessed by the inventor's methods allows for the characterization of differences in the biochemical elements involved in bone density regulation among individuals which further permits individualization of treatment, behavioral modifications, nutritional supplementation (whether customized, i. e., a genetically-guided nutrigenomic product, or generic), and other processes that can limit, inhibit, prevent, or reverse morbidity and mortality associated with undesirable bone density conditions. Most preferably, the intervention is personalized, including a customized nutrigenomic composition. The increased susceptibility occurs whether the assessed polymorphisms are rare or common within certain populations (e.g., ethnic groups) because the methods assess relative susceptibility. It is an essential feature of the invention that at least three (or more, and preferably all) of the claimed polymorphisms be assessed. The number of genes in which the polymorphisms are present is not relevant to the methods described herein, rather it is the contemporaneous assessment of the claimed polymorphisms.

The inventor is the first to recognize that the susceptibility of an individual for undesirable bone density conditions can be assessed by looking at the cumulative effects of the claimed disorder-associated polymorphisms (contemporaneously assessed) in genes whose products have important roles in bone density. Thus, ready assessment of the complicated physiological system that is human bone density can be made by looking at signs of gene dysfunction manifested as the presence of the claimed polymorphisms, even in a human who is asymptomatic of an undesirable bone density condition. The inventor's methods of assessment of the claimed polymorphisms in combination is a better predictor of assessing an individual's susceptibility for an undesirable bone density condition because bone density conditions are multi-genetic pathologies. Maintenance of normal bone density is known to be influenced by the products of several genes. Therefore, assessing the presence of multiple polymorphisms better assesses an individual's global susceptibility to an undesirable bone density condition. Following assessment of the claimed polymorphisms in the human's genome, the selection of an intervention or advisability that an intervention be employed by the human whose genome is assessed can be determined. As noted above, the most preferable intervention includes a customized nutrigenomic nutritional supplement. Susceptibility of a human to an undesirable bone density condition can be used to determine whether the human would benefit by supplementing the human's ordinary nutritional intake with a composition or composition that modulates bone density that contains one or more nutritional supplements or nutriceutical components. Furthermore, relative susceptibility of the human to an undesirable bone density condition can indicate an appropriate dose of such a composition. In another preferable embodiment, suitability of a dietary regimen or intervention for a human can be determined by assessing the human's susceptibility to an undesirable bone density condition. By way of example, small amounts of vitamin D are required in the diet of a healthy human in order to maintain bone density and to regulate bone density in a normal fashion. Amounts significantly greater than the minimum amount required can harm a healthy human, and can, in fact, lead to development of undesirable bone density conditions. The kits and methods disclosed herein can, for example, be used to assess a human's susceptibility to undesirable bone density conditions and identify an appropriate does of vitamin D for administration to an individual, based on that individual's need for, and sensitivity to, vitamin D.

### Description

The invention relates to kits and methods for assessing the relative susceptibility of a human to an undesirable bone density condition by assessing occurrence in the human's genome of genetic polymorphisms that are associated with one or more disorders.

It has been discovered that the degree to which a human is susceptible to an undesirable bone density condition can be assessed by determining which certain polymorphic gene forms (specifically described and claimed herein) are present in the human's genome. The relevant disorder-associated polymorphisms are those which occur in genes which encode products that are involved in bone density regulation and the associated intra- and inter-cellular signaling. Such products include not only parathyroid hormone, parathyroid hormone receptor, calcitonin, and calcitonin receptor, but also can include products which are involved in transmembrane signaling in bone cells, including proteins designated vitamin D receptor, osteocalcin, tumor necrosis factor alpha 1, tumor necrosis factor alpha 1 receptor, the calcium sensing receptor of parathyroid cells, transforming growth factor beta, the alpha 1 subunit of type collagen, other collagen subunits that occur in bone matrix, estrogen receptor alpha, interleukin-6, interleukin-6 receptor, bone morphogenic protein, apolipoprotein E, vitamin D 1 alpha-hydroxylase, insulin growth factor 1, alkaline phosphatase, nucleotide pyrophosphatase, osteocytic membrane calcium transporters (e.g., an L-type voltage operated calcium channel), and parathyroid hormone related protein. Functions of these proteins are described in the art and include signaling the degree or time at which the organic component of bone matrix should be synthesized, signaling the degree or time at which bone matrix mineralization should be promoted or inhibited, signaling the degree or time at which bone demineralization should be promoted or inhibited, signaling the degree or time at which proliferation or activity of osteoblasts should be enhanced, signaling the degree or time at which proliferation or activity of osteoclasts should be enhanced, facilitating transmembrane transmission of these signals, and catalyzing chemical reactions associated with these processes.

It is an essential feature of the invention that at least three or more (and, preferably all) of the polymorphisms identified as a) through j) below are assessed. The polymorphisms assessed by the methods described herein are:

a) occurrence of a cytosine residue in the codon of the gene encoding transforming growth factor beta 1 protein corresponding to amino acid residue 10 of the protein, whereby the codon encodes proline;

b) occurrence of a thymine residue 8 residues upstream of the normal start codon of the gene encoding vitamin D receptor, whereby the residue is part of an initiation codon and the gene encodes a variant vitamin D receptor comprising three additional amino acids at its amino terminus;

c) occurrence of a nucleotide residue that is characteristic of apolipoprotein E polymorphic variant 4;

d) occurrence of a thymine residue in the gene encoding the alpha 1 subunit of type 1 collagen at a site which a guanine usually occurs, whereby a recognition site for the transcription of factor Sp1 is altered;

e) occurrence of a cytosine residue at position −174 of the interleukin 6 gene promoter;

f) occurrence of a guanine residue at the position which a cytosine residue normally occurs in the codon corresponding to amino acid residue 986 of the calcium sensing receptor gene, whereby the codon encodes a serine residue;

g) occurrence of a thymine residue at the position corresponding to position +1417 of the cDNA encoding PtH receptor;

h) occurrence of a thymine residue at the position at which a cytosine residue normally occurs in the codon corresponding to amino acid residue 447 of the calcitonin receptor gene, whereby the codon encodes a leucine residue;

i) occurrence of a thymine residue at amino acid position +1377 of the calcitonin receptor gene; and,

j) occurrence of a cytosine residue where a guanine residue normally occurs at the first nucleotide position of intron 2 of the PtH gene.

The disorder with which a genetic polymorphism in a gene encoding one of the genes described herein is associated need not be a bone density disorder, or even a bone disorder of any type. Association of the polymorphism with any type of disease or disorder is an indication that that polymorphic form of the gene is aberrant and can contribute to osteoporosis, osteopenia, or to another form of an undesirable bone density condition.

Nonetheless, each of the aforementioned genetic polymorphisms assessed by the inventor's methods have been associated with a bone density disorder.

The release of transforming growth factor beta 1 from bone matrix during osteoclastic bone resorption plays an important role in coupling bone formation to bone resorption. Bonewald L. F., et al., Role of transforming growth factor-beta in bone remodeling (1990 Clin Orthop. Relat. Res 250: 261-276). The absence of the polymorphism manifested as occurrence of a cytosine residue in the codon of the gene encoding transforming growth factor beta 1 protein corresponding to amino acid residue 10 of the protein, whereby the codon encodes proline has been associated with a reduced frequency of osteoporotic fractures. Yamada, Y., et al., Association of a polymorphism of the transforming growth factor beta 1 gene with genetic susceptibility to osteoporosis in postmenopausal Japanese women (2000 J.Bone Min.Res. 13:1569-76).

Fok1 polymorphisms, VDR start codon polymorphisms, including the polymorphism manifested as occurrence of a thymine residue 8 residues upstream of the normal start codon of the gene encoding vitamin D receptor, whereby the residue is part of an initiation codon and the gene encodes a variant vitamin D receptor comprising three additional amino acids at its amino terminus, have been associated with bone mineral density, calcium absorption, and peak bone mass. Ames, S. K., et al., Vitamin D Receptor Gene Fokl Polymorphism Predicts Calcuim Absorption and Bone Mineral Density in Children (1999 J.Bone Miner. Res. 14(5):740-6); Harris, S. S., et al., The Vitamin D receptor Start Codon Polymorphism (Fok1) and Bone Mineral Density in Premenopausal American Black and White Women (1997 J.Bone Miner. Res. 12(7): 1043-8).

The ApoE4 polymorphic genotype has been associated with decreased bone metabolism is post-menopausal women. Hayami, Ikeda, et al., Apolipoprotein E phenotype with lipid and bone metabolism after natural menopausal (2001 J.Aichi Med.Univ.Assn. 29(2):83-89).

A polymorphic binding site of the Sp1 transcription factor in the gene encoding the alpha 1 chain of type 1 collagen manifested as occurrence of a thymine residue at a site which a guanine usually occurs, whereby a recognition site for the transcription of factor Sp1 is altered has been significantly associated with bone mineral density. Van Pottelbergh, I., et al., Association of the Type 1 Collagen Alpha 1 Sp1 Polymorphism, Bone Density and Upper Limb Muscle Strength in Community-Dwelling Elderly Men (2001 Osteo.Intl. 12(10) 895-901).

The gene encoding IL-6 plays has long been known to play an important role in bone turnover in laboratory mice. Poli, V., et al., Interleukin-6 deficient mice are protected from bone loss caused by estrogen depletion (1994 Embo.J. 13:1189-96). The G-174C polymorphism in the gene encoding IL-6 has further been associated with bone mineral density and increased fracture risk. Lorentzon, M., et al., Interleukin-6 gene polymorphism is related to bone mineral density during and after puberty in healthy white males: a cross-sectional and longitudinal study (2000 J.Bone Miner. Res. 15(10): 1944-9).

The calcium sensing receptor gene is vital to the regulation of calcium metabolism. Osteoporosis and osteomalacia, commonly known as rickets in children which causes a softening of bones resulting from defective bone mineralization, are calcium metabolism disorders. And, the polymorphism manifested as and alanine to serine substitution at codon 986 of the calcium sensing receptor gene has been associated with hypercalcemia (manifested as an elevated calcium level in the blood) or hypocalcemia (manifested as a deficiency of Vitamin D). Hendy, G., et al., Mutations of the calcium-sensing receptor (CaSR) in familial hypercalcemia, neonatal severe hyperparathyroidism, and autosomal dominant hypocalcemia (2000 Hum.Mut. 16(4): 281-96).

Bone mineral density, and specifically bone mass reduction, has been associated with the polymorphism manifested as the occurrence of a thymine residue at the position corresponding to +1417 of the cDNA encoding parathyroid hormone receptor. Kanzawa, M., et al., Association between parathyroid hormone (PTH)/PTH-related peptide receptor gene polymorphism and the extent of bone mass reduction in primary hyperthyroidism (2000 Horm.Metab.Res. 32(9): 355-8).

The polymorphism manifested as the occurrence of a thymine residue at the position which a cytosine normally occurs in the codon corresponding to amino acid position 447 of the calcitonin receptor gene has been associated with an increased risk for osteoporosis as a result of the proline to leucine substitution. Taboulet, J., et al., Calcitonin receptor polymorphism is associated with decreased fracture risk in postmenopausal women (1998 Hum.Molec.Gen. 7(13): 2129-33).

Occurrence of a thymine residue at amino acid position +1377 of the calcitonin receptor has been associated with significantly lowered lower lumbar bone mineral density in postmenopausal women. Masi, L., et al., Allelic variants of human calcitonin receptor: Distribution and Association with Bone Mass in Postmenopausal Italian Women (1998 Bichem.Biop.Res.Com. 245(2): 622-6).

The polymorphism manifested as occurrence of a cytosine residue where a guanine residue normally occurs at the first nucleotide position of intron 2 of the PtH gene has been correlated with an increased susceptibility to osteitis deformans, also known as Paget's disease, which results in enlarged and deformed bones. Wuyts, L., et al., Evaluation of the Role of RANK and OPG genes in Paget's disease of bone, Bone (2001, 28(1): 104-7).

The genes encoding transforming growth factor beta (TGF-beta), interleukin 6 (IL-6), estrogen receptor alpha (ER-alpha), and vitamin D receptor (VDR) are involved in regulation of bone density. Occurrence of disorder-associated polymorphisms in one or more of these genes should be assessed in the methods described herein, given the importance of these genes. Similarly, the kits described herein preferably include reagents for detecting disorder-associated polymorphisms in one or more of these genes. In addition, the significance of occurrence of disorder-associated polymorphisms in these genes can be applied by assigning a greater weighting factor to disorder-associated polymorphisms of these genes than to disorder-associated polymorphisms in other genes associated with bone density regulation.

Occurrence of disorder-associated polymorphisms in genes encoding products that are involved in a transmembrane signaling pathway in human bone cells (i.e., osteoblasts, osteoclasts, and osteocytes) is also an indication that the human is at risk for developing, or is afflicted with, an undesirable bone density condition. For example, parathyroid hormone (PtH), calcitonin, and their receptors on bone cells are known to be involved in regulation of bone density. In addition to bone cell PtH and calcitonin receptors, such products include other cell surface proteins and integral membrane proteins that are capable of binding extracellular modulators of bone cell activation, bone cell proliferation, bone matrix generation, bone matrix mineralization, and bone matrix resorption. Examples of these proteins include receptors for interleukin-6, vitamin D, tumor necrosis factor alpha 1, estrogen, or systemic calcium. Although these proteins are preferably the proteins that are present on bone cells (except the parathyroid calcium sensing receptor), these proteins can also include the forms that are expressed on other cell types, since those receptors can compete with bone cell receptors for binding of a common ligand. The kits and methods disclosed herein can also be kits and methods that assess occurrence of polymorphisms in a ligand of one of these membrane-associated proteins (e.g., tumor necrosis factor alpha 1, estrogen, or interleukin-6). Many such membrane-associated proteins and corresponding ligands, and their corresponding genes, are known in the art.

Another group of genes for which occurrence therein of a disorder-associated polymorphism is indicative of an enhanced likelihood for, or risk of developing, an undesirable bone density condition are genes which encode a protein for which the level of expression of the protein is associated (i.e., directly or conversely) with a process selected from the group consisting of bone formation, bone erosion, and bone resorption. A disorder-associated polymorphism can result in abnormal expression of the protein products of such genes, thereby perturbing one or more of the processes involved in bone density regulation. For example, the level of expression of the alpha 1 subunit of type 1 collagen (COL1A1) can be altered by a polymorphism in the gene which encodes COL1A1 that results in an altered Sp1 transcription factor recognition site (e.g., as described by Mann et al., 2001, J. Clin. Invest. 107:899-907). Similarly, a polymorphism in the promoter region of the gene which encodes interleukin 6 (IL-6) results in altered IL-6 expression (Ferrari et al., 2001, Arthritis Rheum. 44:196-201). Occurrence of disorder-associated polymorphisms in such genes can provide direct or surrogate indication of the occurrence of, or risk for development of, an undesirable bone density condition in a human.

Another group of genes for which occurrence therein of a disorder-associated polymorphism is indicative of an enhanced likelihood for, or risk of developing, an undesirable bone density condition are genes which encode a protein that forms part of the organic component of bone matrix or which catalyze synthesis of a non-protein part of the organic component. Examples of such proteins include various collagen proteins (e.g., the alpha 1 subunit of type 1 collagen), polypeptides which occur in the ground substance (e.g., various proteoglycans), and enzymes which catalyze generation of a non-polypeptide component of the ground substance (e.g., enzymes involved in synthesis of hyaluronic acid and chondroitin sulfate). Similarly, genes which encode an enzyme which catalyzes deconstruction (i.e., depolymerization and/or cleavage) of the organic part of bone matrix (e.g., collagenases and sulfatases secreted by osteoclasts) can exhibit polymorphic forms, occurrence of which can indicate an enhanced susceptibility to one or more undesirable bone density conditions.

Yet another group of genes for which occurrence therein of a disorder-associated polymorphism is indicative of an enhanced likelihood for, or risk of developing, an undesirable bone density condition are genes which encode a protein that facilitates mineralization or de-mineralization of bone matrix. Examples of such proteins include proteins which facilitate transmembrane transport of mineral components of bone matrix (e.g., calcium, phosphate, magnesium, fluoride, and other ions which occur as mineral salts in bone). Numerous membrane channels, pore-forming proteins, symport, and anti-port proteins which facilitate movement of ions across biological membranes are known, and those which are expressed in membranes of bone cells or in the osteocytic membrane system are preferred for use in the kits and methods disclosed herein.

Given the interaction between vitamin D metabolites (e.g., 1,25-dihydroxycholecalciferol) and calcium flux within cells and across cell membranes, genes which encode enzymes that affect vitamin D metabolism have an important role in bone density regulation. Polymorphisms in these genes can also affect an individual's propensity to develop an undesirable bone density condition. Thus, the kits and methods described herein can also be used to assess occurrence of disorder-associated polymorphisms in genes which encode enzyme which catalyze interconversion of cholecalciferol (vitamin D.sub.3), 25-hydroxycholecalciferol, and 1,25-dihydroxycholecalciferol.

It was not previously appreciated that detection in a human's genome of three or more disorder-associated polymorphisms in genes associated with bone density regulation is indicative that the human globally exhibits enhanced susceptibility to an undesirable bone density condition. Previous studies are believed to have recognized only association between a single polymorphism in one of these genes and a particular disorder. The inventor believes that he is the first to describe methods and kits for assessing a human's global susceptibility to an undesirable bone density condition. This global susceptibility occurs whether the assessed polymorphisms are rare or common within certain populations (e.g., ethnic groups). A human whose genome comprises one or more of the assessed disadvantaged polymorphic alleles should employ an intervention. The advisability that a human employ an intervention increases as the number of assessed disadvantaged polymorphic alleles present in the human's genome increases. Each of the methods described herein may be practiced on individuals who are asymptomatic of an undesirable bone density condition.

Examples of polymorphisms in the foregoing genes which are informative for assessing susceptibility to an undesirable bone density condition include the following:

a) a polymorphism manifested as a change from a thymine to a cytosine in the transforming growth factor beta 1 (TGF-beta 1) coding region which results in a leucine to proline substitution at amino acid position 10 of a TGF-beta 1 polypeptide (Yamada et al., 2001, J. Mol. Med. 79:149-156);

b) a polymorphism manifested as a change from a cytosine to a thymine in the vitamin D receptor (VDR) gene which creates an initiation codon (ATG) three codons proximal to the start site and produces a variant polypeptide comprising three additional amino acids. Gennari, et al., (1999, J. Bone Miner. Res. 14:1379-1386);

c) an apolipoprotein E (apo E) polymorphic variant apolipoprotein E 4 (apo E4);

d) a polymorphism manifested as a change from a guanine to a thymine in the collagen type 1 alpha 1 (COL1A1) gene which alters a recognition site for the transcription factor Sp1. Mann et al. (2001, J. Clin. Invest. 107:899-907);

e) a polymorphism manifested as a change from a guanine to a cytosine at position −174 of the interleukin 6 (IL-6) gene promoter (Ferrari et al., 2001, Arthritis Rheum. 44:196-201);

f) a polymorphism manifested as a change from an alanine to a serine at amino acid position 986 of the calcium sensing receptor (CASR) gene. Cole et al. (2001, Mol. Genet. Metab. 72:168-174);

g) a polymorphism manifested as a change from a cytosine to a thymine at position +1417 of the parathyroid hormone (PTH)/PTH-related peptide (PTHrP) receptor cDNA. Kanzawa et al. (2000, Horm. Metab. Res. 32:355-358);

h) a polymorphism manifested as a change from a thymine to a cytosine in the third intracellular C-terminal domain of the calcitonin receptor gene which results in a proline (CCG) to a leucine (CTG) at amino acid position 447. Taboulet et al. (1998, Hum. Mol. Genet. 7:2129-2133);

i) a polymorphism manifested as a single nucleotide substitution at position +1377 leading to a proline (CCG), leucine (CTG), or heterozygote (C{T/C}G) genotype at amino acid position 463. Nakamura et al. (1997, Hum. Genet. 99:38-41); and,

j) a polymorphism manifested as a guanine to cytosine substitution at the first nucleotide position of intron 2 of the parathyroid hormone (PtH) gene. Hosoi, et al. (1999, Calcif. Tissue. Int. 64:205-208 and in Parkinson, et al. (1992, Nat. Genet. 1:149-152).

Other disorder-associated polymorphisms that occur in genes associated with bone density regulation can be found in the art, and those polymorphisms can be used in the kits and methods described herein in the same manner as those polymorphisms explicitly disclosed herein. Other such polymorphisms include:

a) a polymorphism manifested as a thymine, adenine {(TA).sub.n} repeat located at position −1174 upstream of exon 1 of the human estrogen receptor (ER) gene. Langdahl et al. (2000, J. Bone Miner. Res. 15:2222-2230);

b) a polymorphism manifested as a tetranucleotide simple tandem repeat in intron 4 of the human aromatase cytochrome P-450 gene. Masi, et al., 2001 (J. Clin. Endocrinol. Metab. 86:2263-2269); and,

c) a polymorphism manifested as a cytosine-adenine {(CA).sub.n} repeat between nucleotide positions 947-984 upstream of the transcription start site of the insulin growth factor I (IGF-I) gene. Rosen, et al. (1998 J. Clin. Endocrinol. Metab. 83:2286-2290).

### Methods of Assessing Susceptibility to an Undesirable Bone Density Condition

The invention includes a method of assessing the relative susceptibility of a human to an undesirable bone density condition. This susceptibility can be calculated relative to a hypothetical human whose genome does not contain a single disorder-associated polymorphism in a gene associated with bone formation, bone erosion, or bone resorption. Alternatively, susceptibility can be calculated relative to another human who may have one or more different disorder-associated polymorphisms than the human being assessed. In practice, the basis upon which raw susceptibility scores are calculated is immaterial, so long as the same basis is used for all humans whose scores are to be compared (i.e., so that the scores are relatable to one another). For example, each of the polymorphic alleles present in the human's genome as identified by the inventor's methods can be summed to yield a value for the human. Thereafter, odds ratios can be calculated. Such odds ratios can be calculated based on the number of assessed polymorphic alleles present in the human's genome relative to the total number of alleles. The same calculation can be used to calculate susceptibility relative to another human.

Determining relative susceptibility of a human to an undesirable bone density condition permits assessment of risks and benefits of a variety of compositions, preventive measures, and interventions. In one embodiment, susceptibility of a human to an undesirable bone density condition can be used to determine whether the human would benefit by supplementing the human's ordinary nutritional intake with a composition that contains one or more nutritional supplements or neutriceutical components. Furthermore, relative susceptibility of the human to an undesirable bone density condition can indicate an appropriate dose of such a composition. In another embodiment, suitability of a dietary regimen or intervention for a human can be determined by assessing the human's susceptibility to an undesirable bone density condition. By way of example, small amounts of vitamin D are required in the diet of a healthy human in order to maintain bone density and to regulate bone density in a normal fashion. Amounts significantly greater than the minimum amount required can harm a healthy human, and can, in fact, lead to development of undesirable bone density conditions. The kits and methods disclosed herein can, for example, be used to assess a human's susceptibility to undesirable bone density conditions and identify an appropriate does of vitamin D for administration to an individual, based on that individual's need for, and sensitivity to, vitamin D. As an intervention, a customized composition which addresses the specific genetic deficiencies of the human can be formulated based on the presence of assessed polymorphisms in the human's genome.

Susceptibility of a human to an undesirable bone density condition is assessed by assessing occurrence in the human's genome of a plurality (e.g., 3, 4, 5, 6, 7, 8, or all claimed polymorphisms) of disorder-associated polymorphisms in one or more genes associated with an undesirable bone density condition. Occurrence of an assessed polymorphism in one of these genes is an indication that the human has a greater susceptibility to an undesirable bone density condition than a human in whose genome the polymorphism does not occur. Of course, occurrence of three, four, or more such polymorphisms in the human's genome indicates that the human exhibits even greater susceptibility to an undesirable bone density condition.

Occurrence of every assessed polymorphism in a gene related to a process selected from the group consisting of bone formation, bone erosion, and bone resorption is not necessarily equally indicative of susceptibility to an undesirable bone density condition. In order to account for differences in the significance of various disorder-associated polymorphisms, a weighting factor can be assigned to each polymorphism detected in the methods and kits described herein. As indicated above, certain genes (i.e., those encoding TGF-beta, IL-6, ER-alpha, and VDR) are known to have very significant roles in bone density regulation in humans. All else being equal, disorder-associated polymorphisms that occur in one of these four genes are likely to be more significant than polymorphisms that occur in genes having less significant roles in bone density regulation. Thus, a greater weighting factor can be assigned to polymorphisms that occur in these genes than to others. By way of example, the weighting factor assigned to polymorphisms in these genes can be 1.1 to 10 times (e.g., 2 or 5 times) greater than the weighting factor assigned to disorder-associated polymorphisms (having equal correlation with the corresponding disorder, as discussed below) in other genes. Preferably, the weighting factor assigned to polymorphisms in these is twice that assigned to disorder-associated polymorphisms in other genes.

Another factor which can influence the significance that is assigned to occurrence of a disorder-associated polymorphism in a human's genome is the degree to which the polymorphism is correlated with the corresponding disorder (which, as disclosed above, need not be a bone density disorder or even a bone disorder). Some disorders are highly correlated with occurrence of a genetic polymorphism, and other disorders exhibit lower correlation with a polymorphism. When a polymorphism is reported to be associated with a disorder, a degree of correlation between the polymorphism and the disorder is often reported. One useful way of calculating a factor that describes correlation between a polymorphism and a disorder is to calculate an odds ratio that describes the likelihood that an individual in whose genome the disorder-associated polymorphism occurs will exhibit or develop the disorder. Because the kits and methods described herein can be used to detect whether the human is homozygous for the disease-associated polymorphism, odds ratios calculated for homozygous individuals can also be used, if they are available. Odds ratios can be calculated as described in the art.

For a disorder-associated polymorphism, the odds ratio can be calculated as follows. First, the odds of being afflicted with the disorder are calculated for a first population in whom the polymorphism occurs by dividing the number of afflicted individuals in the first population by the total number of individuals in the first population. Second, the odds of being afflicted with the disorder are calculated for a first population in whom the polymorphism does not occur by dividing the number of afflicted individuals in the second population by the total number of individuals in the second population. Third, the odds ratio is calculated by dividing the odds for the first population by the odds for the second population. If the odds ratio is greater than one, then this is an indication that occurrence of the polymorphism is associated with occurrence of the disorder. Furthermore, the magnitude of the odds ratio is an indication of the significance of the association.

An overall undesirable bone density condition susceptibility score for a human can be determined as follows. A significance score can be assigned to each assessed polymorphism that is detected in the human's genome using a method or kit described herein. The significance score is a constant (e.g., 1.00), and is multiplied by any significance factor (e.g., 1-10, preferably 2 or 5, for the genes encoding calcitonin, a bone cell calcitonin receptor, parathyroid hormone, and a bone cell parathyroid hormone receptor) and by any correlation factor that is available. If information is available which describes the correlation between homozygosity for the polymorphism and the corresponding disorder, then that correlation factor should be used in place of the correlation factor for mere occurrence of the polymorphism, at least if the method or kit is used to rule out occurrence in the subject's genome of corresponding non-disorder-associated polymorphism(s). If significance and correlation factors are not available, then values of 1.00 should be assigned to each. An overall score is determined by summing the significance score for each disorder-associated polymorphism that is detected using the method or kit. This overall undesirable bone density condition susceptibility score can be compared with the values obtained from other subjects, or it can be compared with the value (i.e., zero) which would be expected to occur in a human whose genome does not include any disorder-associated polymorphism in a gene associated with an undesirable bone density condition.

The method used to assess occurrence of any particular disorder-associated polymorphism (or non-disorder-associated polymorphism) is not critical. Numerous methods of detecting occurrence of a polymorphism are known in the art, and substantially any of those methods can be used in the kits and methods described herein. Naturally, the reagents included in the kit will vary depending on the method to be used to detect the polymorphisms. Examples of some suitable polymorphism detection methods are provided below.

In one embodiment, a pair of oligonucleotide primers are used to amplify a portion of the gene that includes a polymorphic region. Detection of one or more of the polymorphisms that occur at the polymorphic region can be achieved by contacting the amplified portion with an oligonucleotide having a sequence such that it will anneal under stringent conditions with the amplified portion only if one polymorphism occurs at the portion, but will not anneal with the amplified portion if another polymorphism occurs at that portion. Various acceptable stringent conditions are known in the art, and can be modified by the skilled artisan as appropriate to any particular amplified portion/oligonucleotide pair. An example of stringent conditions is hybridization in 6.times. sodium chloride/sodium citrate (SSC) at about 45.degree. C., followed by one or more washes in 0.2.times.SSC, 0.1% (w/v) SDS at 65.degree. C.

In an alternative embodiment, one or more molecular beacon oligonucleotides are used to detect polymorphisms (disorder-associated, non-disorder-associated, or both) in a sample that contains a copy of the subject's genome, a fraction of the subject's genome, or amplification products generated from the subject's genome (e.g., amplified portions of genes associated with bone density regulation in which polymorphisms are known to occur).

Molecular beacon probes are single-stranded oligonucleotides having a fluorescent label (e.g., rhodamine, FAM, TET, VIC, JOE, or HEX) attached to one end (e.g., the 5′-end) thereof and a fluorescence quencher (e.g., TAMRA or DABCYL) attached to the other end (e.g., the 3′-end) thereof, as described. Kostrikis, et al. (1998, Science 279:1228-1229). The sequence of each molecular beacon probe is selected to include two complementary hairpin regions, whereby the probe can self-anneal to form a hairpin structure. The 5′- and 3′-ends are brought into close association when the hairpin structure forms. The probe also comprises a targeting portion which is selected to be complementary to a target sequence (e.g., a single polymorphism of a gene associated with bone density regulation). The targeting portion and at least one of the hairpin regions are located in close proximity to one another, meaning that the targeting portion either overlaps the hairpin region or flanks it, having no more than about 5 nucleotide residues therebetween.

If the hairpin regions of the molecular beacon probe anneal with one another, then the probe does not fluoresce, because the hairpin structure forms and the fluorescence quencher attached to one end of the probe quenches fluorescence of the label attached to the other end of the probe. If the targeting portion of the probe anneals with a region of a nucleic acid having the target sequence, then formation of the hairpin structure is inhibited, the fluorescence quencher is not brought into association with the fluorescent label, and the probe fluoresces. Multiple molecular beacon probes can be used in a single reaction mixture, and fluorescence attributable to the probes can be differentiated if the molecular beacon probes are spectrally distinct.

Thus, in this embodiment, one or more molecular beacon probes are used, each having targeting portion which is complementary to a target region (e.g., 20 to 40 nucleotide residues, more preferably 20 to 30 residues) of one polymorphism of a gene associated with bone density regulation (e.g., one of the genes disclosed herein). If the polymorphism to be detected is a single nucleotide polymorphism (SNP), then the target region includes, and preferably is approximately centered around, the nucleotide residue at which the polymorphism occurs. More preferably, two such probes are used, one having a targeting region completely complementary to the target region of one polymorphism of the gene (e.g., one of two polymorphisms of an SNP), and the other having a targeting region completely complementary to the target region of a corresponding polymorphism of the gene (e.g., the other polymorphism of the SNP). Preferably, this pair of probes are spectrally distinct.

In yet another embodiment of how polymorphisms in a gene associated with bone density regulation can be assessed, oligonucleotide primers which are complementary to a region adjacent a characteristic residue of the polymorphism are extended using a polymerase enzyme, and the identity of the nucleotide residue that is added to the primer in the position complementary to the characteristic residue is determined. The primer can be extended in the presence of non-extendable nucleotide residues in order to ensure that a limited number of nucleotide residues (or only one residue) are incorporated into the primer. Methods of this type are known in the art (e.g., the SNP-IT.RTM. technology of Orchid Biocomputer, Inc.) and are described, for example in U.S. Pat. Nos. 6,013,431 and 6,004,744.

### Kits for Assessing Relative Susceptibility to an Undesirable Bone Density Condition

The invention includes a kit for assessing the relative susceptibility of a human to an undesirable bone density condition. The kit contains reagents for performing one or more of the methods described herein. The reagents used in certain embodiments of the methods described herein are indicated above. Reagents useful for performing those methods using a variety of alternative sample preparation and polymorphism detection methods or chemistries are apparent to the skilled artisan.

Kits for detecting polymorphisms in individual genes are known in the art, and the kit of the invention can have similar components. However, a critical feature of the kit is that it includes reagents that permit its user to detect at least two disorder-associated polymorphisms in genes associated with bone density regulation such as the genes described herein (and preferably in one or more of those genes). Preferably the kit includes reagents that permit detection of at least 3, 4, 5, 6, 7, 8 or all disorder-associated polymorphisms in such genes.

In one embodiment, the kit includes a plurality of oligonucleotides which anneal under stringent conditions with a disorder-associated polymorphism of one of the genes, but not with a non-disorder associated-polymorphism. Each of the oligonucleotides is preferably attached to a surface in order to facilitate handling of the oligonucleotide. The oligonucleotides can be linked with a plurality of surfaces (e.g., oligonucleotides for a particular polymorphism being attached to a particle discrete from a particle to which oligonucleotides for another polymorphism are attached), or they can be attached to discrete regions of a single surface (e.g., as in the GENECHIP.TM. device of Affymetrix, Inc.). Annealing between individual oligonucleotides and the polymorphism corresponding thereto can be detected using standard methods. The kit can also comprise oligonucleotides that are useful as molecular beacon probes or as extendable primers.

In one embodiment, the kit further comprises a DNA collection kit or apparatus, such as that described in co-owned U.S. Pat. No. 6,291,171. Advantageously, DNA collected using the kit or apparatus can be stored or archived, and subjected to additional testing as previously unknown polymorphisms are discovered in genes associated with bone density regulation, or as the significance of previously unappreciated polymorphisms is realized.

It will be appreciated by those skilled in the art that changes can made to the embodiments described above without departing from the broad inventive concept thereof.

This invention is not limited to the particular embodiments disclosed, and includes modifications within the spirit and scope of the present invention as defined by the appended claims.

TABLE 1
Advantaged/Disadvantaged Alleles

| | | Disadvantaged |
|---|---|---|
| Gene SNP | Advantaged Allele | Allele |
| Leu10Pro in TGFB1 | Thymine (T) | Cytosine (C) |
| Fok1 in VDR | Cytosine (C) | Thymine (T) |
| APOE4 | (−)/APOE1, APOE2, APOE3 | APOE4 |
| COL1A1 Sp1 | Guanine (G) | Thymine (T) |
| −174C in IL-6 | Guanine (G) | Cytosine (C) |
| C986G in CaSR | Cytosine (C) | Guanine (G) |
| C1417T in PtH | Cytosine (C) | Thymine (T) |
| C447T in CaSR | Cytosine (C) | Thymine (T) |
| C1377T in CTR | Cytosine (C) | Thymine (T) |
| C→G PtH Intron 2 | Guanine (G) | Cytosine (C) |

What is claimed is:

1. A method of assessing the advisability that a human should employ an intervention, the method comprising assessing occurrence in the human's genome of at least three polymorphisms selected from the group consisting of
a) occurrence of a cytosine residue in the codon of the gene encoding transforming growth factor beta 1 protein corresponding to amino acid residue 10 of the protein, whereby the codon encodes proline;
b) occurrence of a thymine residue 8 residues upstream of the normal start codon of the gene encoding vitamin D receptor, whereby the residue is part of an initiation codon and the gene encodes a variant vitamin D receptor comprising three additional amino acids at its amino terminus;
c) occurrence of a nucleotide residue that is characteristic of apolipoprotein E polymorphic variant 4;
d) occurrence of a thymine residue in the gene encoding the alpha 1 subunit of type 1 collagen at a site which a guanine usually occurs, whereby a recognition site for the transcription of factor Sp 1 is altered;
e) occurrence of a cytosine residue at position −174 of the interleukin 6 gene promoter;
f) occurrence of a guanine residue at the position which a cytosine residue normally occurs in the codon corresponding to amino acid residue 986 of the calcium sensing receptor gene, whereby the codon encodes a serine residue;
g) occurrence of a thymine residue at the position corresponding to position +1417 of the cDNA encoding PtH receptor;
h) occurrence of a thymine residue at the position at which a cytosine residue normally occurs in the codon corresponding to amino acid residue 447 of the calcitonin receptor gene, whereby the codon encodes a leucine residue;
i) occurrence of a thymine residue at amino acid position +1377 of the calcitonin receptor gene; and,
j) occurrence of a cytosine residue where a guanine residue normally occurs at the first nucleotide position of intron 2 of the PtH gene,
wherein the advisability that the human should employ an intervention is determined based on occurrence of the assessed polymorphisms.

2. The method of claim 1, comprising assessing occurrence in the human's genome of at least five of a) through j).

3. The method of claim 1, comprising assessing occurrence in the human's genome of each of a) through j).

4. The method of claim 1, wherein the human is asymptomatic of an undesirable bone density condition.

5. The method of claim 1, the method thereby determining whether the human is predisposed to developing an undesirable bone density condition.

6. The method of claim 1, further comprising providing an instructional material to the human.

7. The method of claim 1, wherein the intervention is a customized nutrigenomic composition.

8. A method for determining whether a human is predisposed to developing an undesirable bone density condition, the method comprising assessing occurrence in the human's genome of at least three polymorphisms selected from the group consisting of
a) occurrence of a cytosine residue in the codon of the gene encoding transforming growth factor beta 1 protein corresponding to amino acid residue 10 of the protein, whereby the codon encodes proline;
b) occurrence of a thymine residue 8 residues upstream of the normal start codon of the gene encoding vitamin D receptor, whereby the residue is part of an initiation codon and the gene encodes a variant vitamin D receptor comprising three additional amino acids at its amino terminus;
c) occurrence of a nucleotide residue that is characteristic of apolipoprotein E polymorphic variant 4;
d) occurrence of a thymine residue in the gene encoding the alpha 1 subunit of type 1 collagen at a site which a guanine usually occurs, whereby a recognition site for the transcription of factor Sp 1 is altered;
e) occurrence of a cytosine residue at position −174 of the interleukin 6 gene promoter;
f) occurrence of a guanine residue at the position which a cytosine residue normally occurs in the codon corresponding to amino acid residue 986 of the calcium sensing receptor gene, whereby the codon encodes a serine residue;
g) occurrence of a thymine residue at the position corresponding to position +1417 of the cDNA encoding PtH receptor;
h) occurrence of a thymine residue at the position at which a cytosine residue normally occurs in the codon corresponding to amino acid residue 447 of the calcitonin receptor gene, whereby the codon encodes a leucine residue;
i) occurrence of a thymine residue at amino acid position +1377 of the calcitonin receptor gene; and,
j) occurrence of a cytosine residue where a guanine residue normally occurs at the first nucleotide position of intron 2 of the PtH gene,
wherein presence of the assessed polymorphisms indicates that the human is predisposed to developing an undesirable bone density condition.

9. The method of claim 8, comprising assessing occurrence in the human's genome of at least five of a) through j).

10. The method of claim 8, comprising assessing occurrence in the human's genome of each of a) through j).

11. The method of claim 8, wherein the human is asymptomatic of an undesirable bone density condition.

12. The method of claim 8, the method further comprising providing an instructional material to the human.

13. The method of claim 8, the method further comprising advising that the human employ an intervention.

14. The method of claim 14, wherein the intervention is a customized nutrigenomic composition.

15. A method comprising assessing the degree to which a human is susceptible to an undesirable bone density condition by identifying at least three polymorphisms selected from the group consisting of
a) occurrence of a cytosine residue in the codon of the gene encoding transforming growth factor beta 1 protein corresponding to amino acid residue 10 of the protein, whereby the codon encodes proline;
b) occurrence of a thymine residue 8 residues upstream of the normal start codon of the gene encoding vitamin D receptor, whereby the residue is part of an initiation codon and the gene encodes a variant vitamin D receptor comprising three additional amino acids at its amino terminus;
c) occurrence of a nucleotide residue that is characteristic of apolipoprotein E polymorphic variant 4;
d) occurrence of a thymine residue in the gene encoding the alpha 1 subunit of type 1 collagen at a site which a guanine usually occurs, whereby a recognition site for the transcription of factor Sp1 is altered;
e) occurrence of a cytosine residue at position −174 of the interleukin 6 gene promoter;
f) occurrence of a guanine residue at the position which a cytosine residue normally occurs in the codon corresponding to amino acid residue 986 of the calcium sensing receptor gene, whereby the codon encodes a serine residue;
g) occurrence of a thymine residue at the position corresponding to position +1417 of the cDNA encoding PtH receptor;
h) occurrence of a thymine residue at the position at which a cytosine residue normally occurs in the codon corresponding to amino acid residue 447 of the calcitonin receptor gene, whereby the codon encodes a leucine residue;
i) occurrence of a thymine residue at amino acid position +1377 of the calcitonin receptor gene; and,
j) occurrence of a cytosine residue where a guanine residue normally occurs at the first nucleotide position of intron 2 of the PtH gene,
thereafter calculating a susceptibility value for the condition by summing the number of polymorphic alleles identified in the human's genome to yield a value for the human, wherein a value greater than zero indicates a greater susceptibility to the undesirable bone density condition for the human, the method thereby allowing determination of the degree to which the human is susceptible to the undesirable bone density condition relative to another human.

16. The method of claim 15, comprising assessing occurrence in the human's genome of at least five of a) through j).

17. The method of claim 15, comprising assessing occurrence in the human's genome of each of a) through j).

18. The method of claim 15, the method further comprising advising that the human employ an intervention.

19. The method of claim 18, wherein the intervention is a customized nutrigenomic composition.
